(19) United States
(12) Patent Application Publication
(10) Pub. No.: US 2013/0338165 A1
(43) Pub. Date: Dec. 19, 2013

(54) Use of Compounds Elevating Glutathione Levels for the Treatment of Parkinson's Disease

(71) Applicant: Promentis Pharmaceuticals, Inc., Milwaukee, WI (US)

(72) Inventors: David A. Baker, Grafton, WI (US); Daniel G. Lawton, Bayside, WI (US); Chad Beyer, Englewood, CO (US); Michael Neary, West Allis, WI (US)

(21) Appl. No.: 13/919,768

(22) Filed: Jun. 17, 2013

Related U.S. Application Data
(60) Provisional application No. 61/660,381, filed on Jun. 15, 2012.

(51) Int. Cl. A61K 31/198

(52) U.S. Cl. CPC A61K 31/198
USPC 514/252.11; 514/252.1; 514/369; 514/383; 514/482; 514/513; 514/547; 514/548; 514/533; 514/562

(57) ABSTRACT

Methods of treating Parkinson's Disease comprising administering compounds that up-regulate cystine-glutamate exchange (system X_{c}⁻) and/or increase glutathione levels in the brain cells. Preferred compounds are cysteine/cystine prodrugs or N-acetyl cysteine (NAC) prodrugs.

### FIELD OF THE INVENTION

This invention relates to the use of compounds that up-regulate cystine-glutamate exchange (system X_{c}⁻) and/or increase glutathione levels in the brain cells for the treatment of Parkinson's disease.

### BACKGROUND OF THE INVENTION

Investigation into discovering novel therapeutic strategies that address the underlying pathophysiology of Parkinson's disease has revealed that this neurodegenerative disorder is characterized by the progressive loss of dopamine neurons in discrete regions of the brain. More specifically, this cellular damage occurs primarily in the substantia nigra, a dopamine-rich area of the central nervous system (CNS) responsible for orchestrating motor control and coordination—disturbances of which are hallmark clinical features of Parkinson's disease. Several biochemical markers of oxidative stress, including damage to DNA and destruction of cellular macromolecules, protein formation and lipid peroxidation are all elevated in patients with Parkinson's disease, while a significant decrease in the levels of antioxidants such as glutathione are reported to occur during the progression of the disease. Moreover, histopathologies directly linked to damage caused by the formation of reactive oxygen species (ROS), oxidative stress, and subsequent mitochondrial dysfunction, are found in patients with Parkinson's disease.

There are currently no effective drugs or methods of treating Parkinson's disease. Accordingly, there is a significant need for new therapeutical agents to treat Parkinson's disease.

### SUMMARY OF THE INVENTION

The invention provides methods of treating Parkinson's disease comprising administering to a patient in need thereof a therapeutically effective amount of a compound that up-regulates cystine-glutamate exchange (system X_{c}⁻) and/or increases glutathione levels in the brain cells. In some embodiments, the compound suitable for the purposes of the invention is a cysteine/cystine prodrug or an N-acetyl cysteine (NAC) prodrug.

The compounds that are believed to be effective for the treatment of Parkinson's disease, include but are not limited to all of the compounds disclosed and/or claimed in the following patents and patent applications, the disclosures of which are hereby incorporated by reference in their entirety: U.S. Pat. Nos. 7,829,709 and 8,173,809; US Patent Application Publication Numbers 2011/0021533 A1; US 2010/0048587 A1; US 2011/0224156 A1; US 2012/0122793 A1; US 2012/0122792 A1; US 2012/0220596; and PCT International Application Publication Number WO/2013/016727.

In a preferred embodiment, the methods of the present invention encompass administering the following compounds:

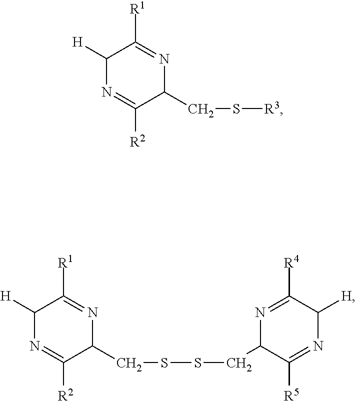

wherein R¹, R², R⁴ and R⁵ are independently selected from OH, ═O, or a branched or straight chain C₁ to C₅ alkoxy group,

with the caveats that when ═O is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O; and

R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group;

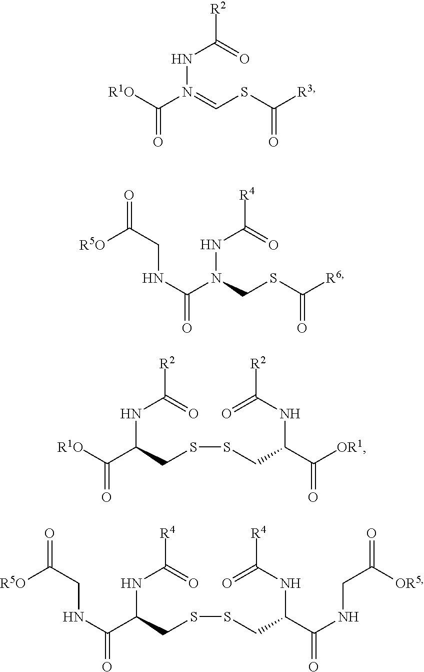

wherein R¹ through R⁶ are independently selected from a branched or straight chain C₁ to C₅ alkyl, a phenyl, or a benzyl group;

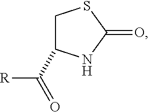

wherein R is selected from the group consisting of:

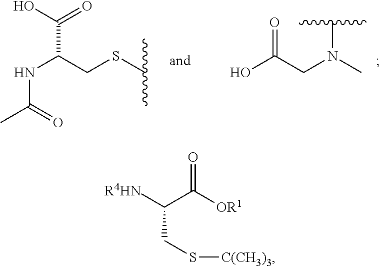

wherein

R¹ is selected from the group consisting of CH₃, CH₂Cl₃, CH(CH₃)₂, CH₂-phenyl, and phenyl;

R⁴ is selected from the group consisting of H, C(O)R₂, and

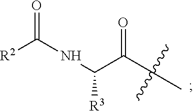

R² is selected from the group consisting of CH₃, CH₂CH₃, CH(CH₃)₂, CH₂-phenyl, and phenyl; and

R³ is selected from the group consisting of H, CH₃, CH₂-phenyl, CH(CH₃)₂, CH₂OH,

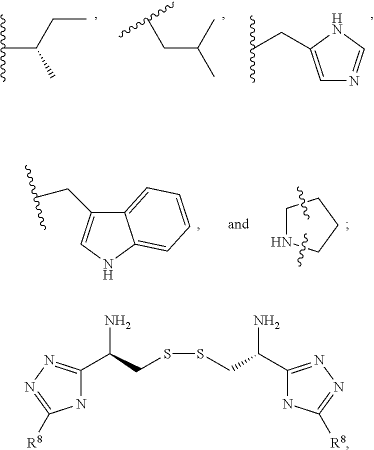

wherein R⁸ is selected from the group consisting of H, CH₃, CH₂CH₃, CH(CH₃)₂ and phenyl.

The invention also encompasses pharmaceutically acceptable salts, esters, bioisosteres, enantiomers, diastereoisomers, mixtures of enantiomers/diastereoisomers, and prodrugs of the provided compounds,

In one embodiment, the methods of the present invention encompass administering the following compound:

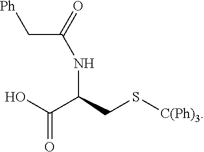

### DETAILED DESCRIPTION OF THE INVENTION

### Definitions

The following definitions are used, unless otherwise described.

The term “prodrugs” refers to compounds, including but not limited to monomers and dimers of the compounds useful for the purposes of the invention, which become under physiological conditions compounds useful for the purposes of the invention or the active moieties of the compounds useful for the purposes of the invention.

The term “active moieties” refers to compounds which are pharmaceutically active in vivo, whether or not such compounds are compounds useful for the purposes of the invention.

The term “composition” is intended to encompass a product comprising the specified ingredients in the specified amounts, as well as any product which results, directly or indirectly, from a combination of the specified ingredients in the specified amounts.

The term “subject” includes mammals, including humans. The terms “patient” and “subject” are used interchangeably.

In general, unless indicated otherwise, a chemical group referred to anywhere in the specification can be optionally substituted.

The term “therapeutically effective amount” means the amount of a compound that, when administered to a subject for treating Parkinson's disease, is sufficient to effect such treatment for the Parkinson's disease. Treating of the Parkinson's disease does not require the achievement of complete cure. The “therapeutically effective amount” can vary depending on the variety of factors, including the compound, the severity of the Parkinson's disease; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

In one embodiment, the terms “treating” or “treatment” refer to ameliorating the Parkinson's disease (i.e., arresting or reducing the development of the Parkinson's disease or at least one of the clinical symptoms thereof). In another embodiment, “treating” or “treatment” refers to ameliorating at least one physical parameter, which may not be discernible by the subject. In yet another embodiment, “treating” or “treatment” refers to modulating the Parkinson's disease, either physically, (e.g., stabilization of a discernible symptom), physiologically, (e.g., stabilization of a physical parameter), or both. In yet another embodiment, “treating” or “treatment” refers to delaying the onset of the Parkinson's disease, or even preventing the same.

The term “combinational use” as used in the present invention encompasses co-formulations of the two active agents as well as co-administration of two active agents as separate formulations

### Description of the Invention

The present invention incorporates and is based on new and emerging scientific understanding of Parkinson's disease.

In its broadest embodiment, the invention provides methods of treating Parkinson's disease comprising administering to a patient in need thereof a therapeutically effective amount of a compound that up-regulates cystine-glutamate exchange (system X_{c}⁻) and/or increases glutathione levels in the brain cells. In some embodiments, the compound suitable for the purposes of the invention is a cysteine/cystine prodrug or an N-acetyl cysteine (NAC) prodrug.

The compounds that are believed to be effective for the treatment of Parkinson's disease, include but are not limited to all of the compounds disclosed and/or claimed in the following patents and patent applications, the disclosures of which are hereby incorporated by reference in their entirety: U.S. Pat. Nos. 7,829,709 and 8,173,809; US Patent Application Publication Numbers 2011/0021533 A1; US 2010/0048587 A1; US 2011/0224156 A1; US 201210122793 A1; US 2012/0122792 A1: US 2012/0220596; and POT International Application Publication Number WO/2013/016727.

Several pieces of evidence suggest a neuroprotective role for glutathione and its common precursor, N-acetylcysteine (NAC), in Parkinson's disease. One of the earliest biochemical processes observed in patients with Parkinson's disease is a significant decrease (40% compared to controls) in glutathione levels. Such depletions in glutathione, and in particular to its antioxidant activity, are suggested to contribute to increased oxidative stress in patients with Parkinson's disease. Moreover, NAC, despite its weak penetration into the CNS, has been shown to counter age-related mitochondrial damage, prevent apoptosis, and scavenge hydrogen peroxide and reactive quinones. Given these findings, it is suggested that therapeutic strategies designed to efficiently elevate glutathione levels should have profound therapeutic value for patients suffering from Parkinson's disease. Moreover, this could best be achieved with a cysteine pro-drug that exhibits superior CNS permeability relative to NAC.

Cystine-glutamate exchange (system x_{c}−) is ubiquitously expressed throughout the body and brain and regulates the direct exchange of extracellular cystine for intracellular glutamate. A primary function of system x_{c}− has been identified as providing cells with cystine used in the synthesis of glutathione, the body's main antioxidant and potent free radical scavenger, protecting cells from damage induced by ROS formation. A second and equally important role of system x_{c}− is regulating a critical source of extracellular glutamate capable of stimulating extrasynaptic receptors and modulating synaptic release of neurotransmitters, including dopamine and glutamate.

Mitochondrial dysfunction, oxidative stress and nigrostriatal toxicity are proposed to result, in large part, from diminished glutathione levels. It has previously been demonstrated that depletion in glutathione is linked to an increase in system protein, at least in rats receiving unilateral 6-hydroxydopamine lesions. Additionally, NAC, which is a molecule known to engage system x_{c}−, combats age-related mitochondrial damage, prevents apoptosis and scavenges a variety of ROS under numerous experimental conditions.

Further, in animal models of Parkinson's disease, NAG has exhibited therapeutic potential in mice treated with 1-methyl-4-phenyl-1,2,3,6-tetrahydropyridine (MPTP) and in alpha-synuclein overexpressing mice. Moreover, NAG provides protection against oxidative stress in the brain of rats and gerbils following systemic administration.

A brain permeable version of NAC (known as AD4) also protected cells from neurotoxicity induced by rotenone. Finally, in clinical experiments, despite having very modest CNS permeability, intravenous administration of glutathione itself reduced motor impairment and disability by 40%.

Based on these studies, the inventors of the present application reasonably believe that increasing system x_{c}− activity and increasing glutathione levels may be clinically effective for treating Parkinson's disease in a manner that may target an important underlying pathophysiological process. Further, pharmacological strategies designed to ultimately reduce or prevent the formation of ROS, may have profound disease modifying potential for patients with Parkinson's disease.

The efficacy of these compounds for the treatment of Parkinson's disease may be confirmed through pharmacodynamic studies and through utilizing a standard MPTP model of Parkinson's disease as explained in more detail in the Examples section of this application.

The compounds disclosed and/or claimed in U.S. Pat. Nos. 7,829,709 and 8,173,809; US Patent Application Publication Numbers 2011/0021533 A1; US 2010/0048587 A1; US 2011/0224156 A1; US 2012/0122793 A1; US 2012/0122792 A1; U.S. patent application No. 13/465,383 filed May 7, 2012; and U.S. Provisional Patent Application No. 61/512,751, filed Jul. 28, 2011 are either cysteine/cystine prodrugs and/or NAG prodrugs and/or the compounds that increase glutathione levels. Accordingly, this invention provides methods of using these compounds for the treatment of Parkinson's disease comprising administering to a patient in need thereof a therapeutically effective amount of one or more of these compounds.

Specifically, these compounds are believed to be superior to glutathione and NAC for the treatment of Parkinson's disease because glutathione and NAC cannot be used effectively in the clinic because they do not readily enter the CNS and possess poor solubility characteristics.

In a preferred embodiment, the methods of the present invention encompass administering the following compounds:

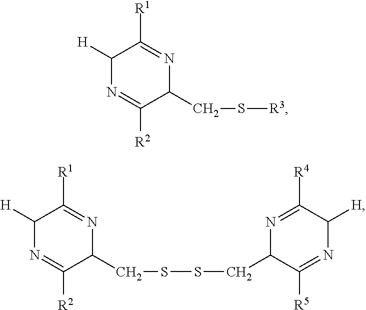

wherein R¹, R², R⁴ and R⁵ are independently selected from OH, ═O, or a branched or straight chain C₁ to C₅ alkoxy group,

with the caveats that when αO is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O; and

R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group;

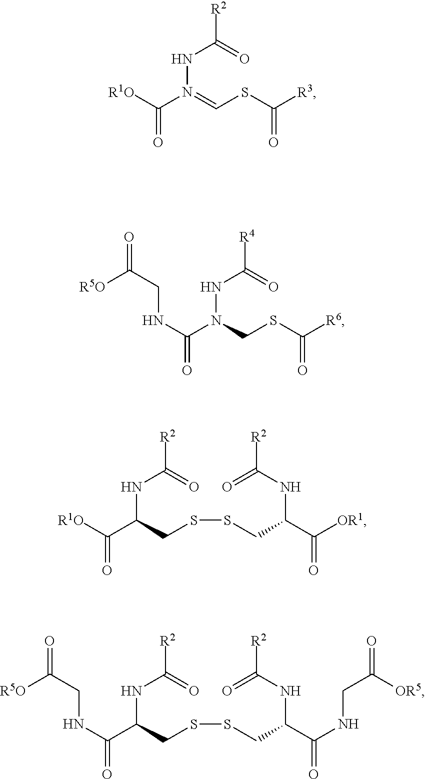

wherein R¹ through R⁶ are independently selected from a branched or straight chain C₁ to C₅ alkyl, a phenyl, or a benzyl group;

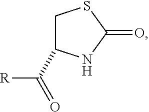

wherein R is selected from the group consisting of:

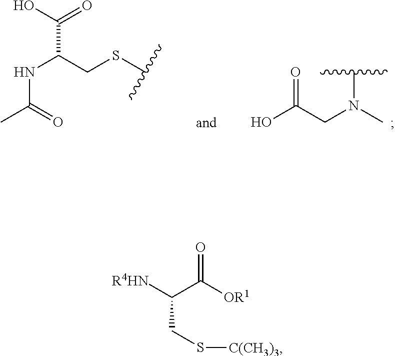

wherein

R¹ is selected from the group consisting of CH₃, CH₂CH₃, CH(CH₃)₂, CH₂-phenyl, and phenyl;

R⁴ is selected from group consisting of H, C(O)R₂, and

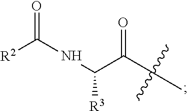

R² is selected from the group consisting of CH₃, CH₂CH₃, CH(CH₃)₂, CH₂-phenyl, and phenyl; and

R³ is selected from the group consisting of H, CH₃, CH₂-phenyl, CH(CH₃)₂, CH₂OH,

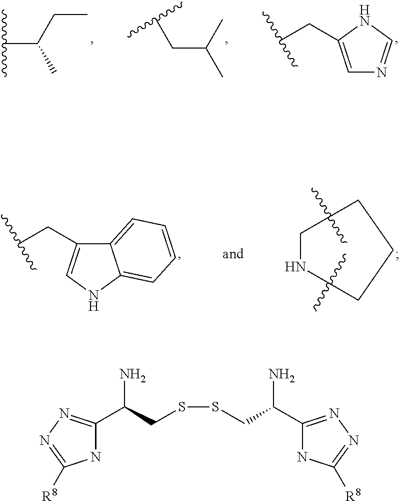

wherein R⁸ is selected from the group consisting of H, CH₃, CH₂CH₃, CH(CH₃)₂ and phenyl.

The invention also encompasses pharmaceutically acceptable salts, esters, bioisosteres, enantiomers, diastereoisomers, mixtures of enantiomers/diastereoisomers, and prodrugs of the provided compounds.

In one embodiment, the methods of the present invention encompass administering the following compound:

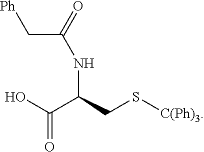

On the basis of specific parameters, this compound is believed to be best suited to cross into the CNS.

Using an in vitro screening assay conducted in human glial cells from brain astrocytoma (1321N1), a cell line with high system x_{c}− expression, the compound with the following formula:

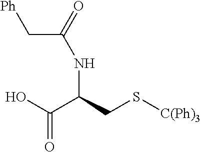

was found to drive system x_{c}− as evidenced by a robust and significant decrease in the uptake of ¹⁴C-cystine and significant elevations in ³H-glutamate.

In efficacy models of antipsychotic-like and anxiolytic-like activity (pre-pulse inhibition (PPI) and elevated plus maze (EPM), respectively), this compound is orally-active and produces significant behavioral effects in both of these models. Further, in the PPI model, this compound elicits an antipsychotic-like response (at a comparable dose range) similar to the commercially-available antipsychotic clozapine.

On the basis of these data, and additional in vitro selectivity experiments at nearly 50 separate enzymes, transporters, and GPCRs (data not shown), the inventors believe that the most preferred compounds exemplified by the structural formulas above increase system x_{c}− activity and increase glutathione levels, and therefore are suitable for the treatment of Parkinson's disease. Furthermore, lead compounds from these representative structures above, have demonstrated activity at system x_{c}− as evidenced by a robust and significant decrease in the uptake of ¹⁴C-cystine and significant elevations in glutamate release and therefore, are suitable for the treatment of Parkinson's disease.

The present invention also provides pharmaceutical compositions that comprise the compounds suitable for the purposes of the present invention formulated together with one or more non-toxic pharmaceutically acceptable carriers. The pharmaceutical compositions can be specially formulated for oral administration in solid or liquid for for parenteral injection or for rectal administration.

The pharmaceutical compositions of this invention can be administered to humans and other mammals orally, rectally, parenterally, intracisternally, intravaginally, transdermally (e.g. using a patch), transmucosally, sublingually, pulmonary, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

In another aspect, the present invention provides a pharmaceutical composition comprising a compound suitable for the purposes of the present invention and a physiologically tolerable diluent. The present invention includes one or more compounds as described above formulated into compositions together with one or more non-toxic physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microctstalline cellulose, aluminum metahydroxide, bentonite, agar-agar and tragacanth, or mixtures of these substances, and the like.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds suitable for the purposes of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound suitable for the purposes of this invention include powders, sprays, ointments and inhalants. The active compound is mixed under sterile conditions with a pharmaceutically acceptable carrier and any needed preservatives, buffers or propellants which can be required. Ophthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the Parkinson's disease and prior medical history of the patient being treated. However, it is within the skill of the art to start doses of the compound at levels lower than required to achieve the desired therapeutic effect and to gradually increase the dosage unto the desired effect is achieved.

When used in the above or other treatments, a therapeutically effective amount of one of the compounds suitable for the purposes of the present invention can be employed in pure form or, where such forms exist, in pharmaceutically acceptable salt, ester or prodrug form. Alternatively, the compound can be administered as a pharmaceutical composition containing the compound of interest in combination with one or more pharmaceutically acceptable excipients.

The total daily dose of the compounds suitable for the purposes of this invention administered to a human or lower animal may range from about 0.0001 to about 1000 mg/kg/day. If desired, the effective daily dose can be divided into multiple doses for purposes of administration; consequently, single dose compositions may contain such amounts or submuitiples thereof to make up the daily dose.

The methods of the invention can be used in combination with the use of other drugs known for the treatment of Parkinson's Disease, including but no limited to, levo-dopa (L-DOPA), zonasomide and others.

For a clearer understanding of the invention, details are provided below. These are merely illustrations and are not to be understood as limiting the scope of the invention in any way. Indeed, various modifications of the invention in addition to those shown and described herein will become apparent to those skilled in the art from the following examples and foregoing description. Such modifications are also intended to fall within the scope of the appended claims.

### EXAMPLES

### Example 1

### Evaluation of Lead Molecule in MPTP Model of Parkinson's Disease (Prophetic)

After scaling up the drug candidate, we propose to demonstrate preclinical proof-of-efficacy by utilizing a standard mouse MPTP model of Parkinson's disease. In the first set of studies, our lead molecule will be evaluated at 3 doses in the mouse MPTP model (eight week old male C57/B6. The following treatment groups will be used: MPTP/Vehicle (N=10), MPTP/drug dose 1 (N=10), MPTP/drug dose 2 (N=10), MPTP/drug dose 3 (N=10), Vehicle/drug dose 1 (N=10), Vehicle/drug dose 2 (N=10), Vehicle/drug dose 3 (N=10), Untreated controls (N=6). In these studies, MPTP-HCl (20 mg/kg) will be administered by subcutaneous injection, twice daily (4 hrs apart) for 5 consecutive days. Our lead drug will be administered orally 30 minutes prior to each MPTP injection and once daily for 3 weeks following the last MPTP exposure. Based on secondary in vitro selectivity data generated on our lead molecules, these compounds do not appear to have affinity for the dopamine transporter (data not shown) and therefore are not expected to interfere with the conversion of MPTP to 1-methyl-4-phenylpyridinium ion (MPP⁺). However, to confirm a lack of test drug effect on striatal MPP⁺ accumulation, a separate group of animals (N=6) will be administered MPTP as described above with test drug (dose to be determined) 30 min before each MPTP injection and at the end of the 5 day MPTP administration period, this group of animals will be euthanized and the striatum removed for measurement of MPP+ levels. There is also a potential concern that the lead compound, considering its capability of stimulating extrasynaptic receptors and modulating synaptic release of neurotransmitters, including glutamate, may have undesired excitotoxic effects (although this potential concern is decreased—perhaps dramatically—with reference to the potential that driving Xc− will actually decrease synaptic glutamate and as a result prevent excitotoxicity).

The possibility of drug-induced toxicity will be assessed in the vehicle/drug groups and data compared against untreated control animals. As described in detail below, striatal tissue from these animals will be assessed for dopamine and metabolite levels and the substantia nigra will be examined for any possible toxicity by performing counts of both tyrosine hydroxylase-positive neurons (which will tell us if there is a specific dopaminergic toxicity) and counts of cresyl violet stained neurons (which will tell us if these is any non-specific toxic effects, including excitotoxicity),

Additional studies will be conducted to determine the extent to which neuroprotection may be achieved using a delayed administration of the lead drug candidate. The following treatment groups will be used: MPTP/Vehicle (N=10), MPTP/drug dose 1 (N=10), MPTP/drug dose 2 (N=10), MPTP/drug dose 3 (N=10), Untreated controls (N=6). In these studies, MPTP-HCl (20 mg/kg) will be administered by subcutaneous injection, twice daily (4 hrs apart) for 5 consecutive days, as discussed above. For these studies, the lead drug will be administered orally once daily for 3 weeks, beginning 24 hours after the last MPTP injection. Since NAC \is a molecule known to engage system x_{c}−, NAC has been suggested to have a neuroprotective effect in an acute MPTP model and in synuclein overexpressing mice, and we propose that our lead compound will have a superior effect compared to NAC, we will directly assess the effects of NAC in the MPTP mouse model described in this application as a comparator to our lead drug. The following treatment groups will be used: MPTP/Vehicle (N=10), MPTP/NAC (30 mg/kg, i.p. (N=10), MPTPINAC (500 mg/kg, i.p. (N=10), Untreated controls (N=6), In these studies, MPTP-Hcl (20 mg/kg) will be administered by subcutaneous injection, twice daily (4 hours apart) for 5 consecutive days, as discussed above. For these studies, the NAC will be administered 3 hours before each MPTP injection will be administered once daily for 3 weeks, following the last MPTP injection. These doses and timing of NAC administration are based on data showing a partial neuroprotective effect of NAC in a different MPTP mouse models (Aoyama et al., 2008; Perry et al., 1985).

### Behavioral Measurements

The number of rearings will be assessed as a behavioral measurement for these studies. Specifically, in a plastic cylinder (13 cm in diameter, 16 cm height), mice will be allowed to explore freely for 2 min. Rears will be counted as elevations to an erect stance, and separated by forelimb contact to the horizontal base of the cylinder. Free-standing rears (FSR), wall-assisted rears (WAR) and total rears will be counted. Mice will not be acclimated to the cylinder prior to baseline testing. Behavioral data will be collected at baseline and at the end of the study.

### Biochemical Measurements

At the conclusion of the study, animals will be euthanized by decapitation, the striatum will be removed fresh and flash frozen for later analysis of dopamine (DA) and metabolite levels by HPLC. Briefly, tissue will be sonicated in 0.4M perchloric acid and centrifuged at 15000 rpm for 5 minutes at 4° C. Supernatant will be removed for analysis by HPLC as previously described, using isoproterenol as an internal standard. Samples will then be analyzed using a Coulochem Ill HPLC system with an electrochemical detector (ESA, Inc). Peak heights will be compared with internal standard values to determine the concentration DA and its metabolites (EZchrome V3.1, Agilent Technologies).

### Histological Measurements

After the striatum is dissected, the remaining tissue will be post-fixed in 4% paraformaldehyde for 72 hr for histological analysis. Fixed tissue blocks will be immersed in 30% sucrose as a cryo-protectant and sectioned frozen on a sliding microtome (30 μm section thickness) through the rostro-caudal extent of the substantia nigra pars compacta. Every third section will be processed for tyrosine hydroxylase (TH) immunohistochemistry (rabbit anti-TH, 1:1000, Pel-freez) and adjacent sections will be stained with cresyl violet. Cells (both TH⁺ and cresyl violet stained (Nissl⁺)) will be counted using unbiased stereology (StereoInvestigator, MBFbioscience). The region of interest will be outlined under low magnification (4×) and a grid measuring 195 μm×85 μm will be randomly placed over the region. Cells will be then counted at high power (100×) using a counting frame measuring 40 μm². A cell will be counted only if a nucleus is clearly identifiable and the cell is completely within the counting frame. This process will be repeated for each section in the series for a given animal and a total of 10 total sections/animal will be analyzed.

What is claimed is:

1. A method of treating Parkinson's disease comprising administering to a patient in need thereof a therapeutically effective amount of a compound that up-regulates cystine-glutamate exchange (system X_{c}⁻) and increases glutathione levels in the brain cells of said patient.

2. The method of claim 1, wherein said compound is a cysteine/cystine prodrug or an N-acetyl cysteine (NAC) prodrug.

3. The method of claim 1, wherein said compound is selected from the group consisting of 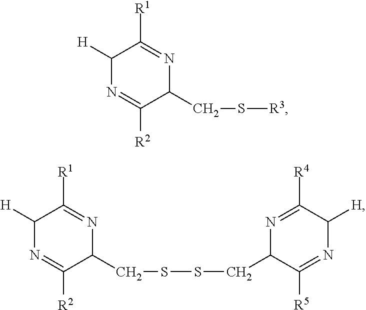
wherein R¹, R², R⁴ and R⁵ are independently selected from OH, ═O, or a branched or straight chain C₁ to C₅ alkoxy group,
with the caveats that when ═O is selected the nitrogen atom adjacent the carbonyl group thusly formed bears a H and a single bond joins the adjacent nitrogen to said carbonyl group and further that the R¹, R², R⁴ and R⁵ that appear in the structure shall be selected to not all be ═O; and
R³ is H, a branched or straight chain C₁ to C₅ alkyl, a nitrobenzenesulfonyl, an aryl thio, an aryl, an alkylthio, an acyl, a benzoyl, a thio acyl, a thio benzoyl, or a benzyl group; 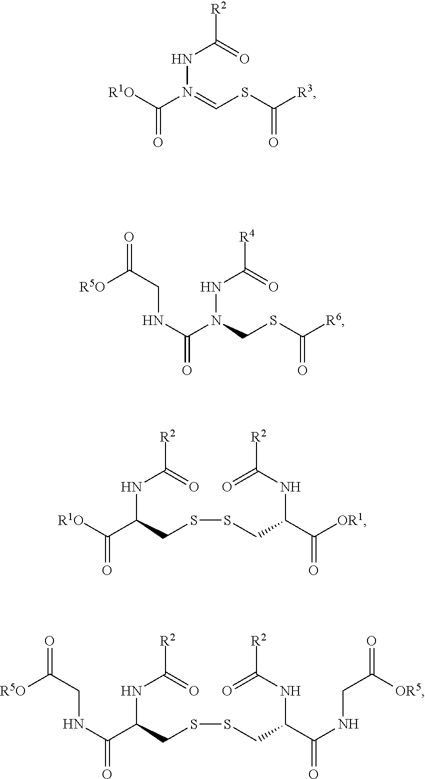
wherein R¹ through R⁶ are independently selected from a branched or straight chain C₁ to C₅ alkyl, a phenyl, or a benzyl group; 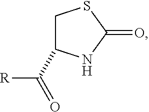
wherein R is selected from the group consisting of: 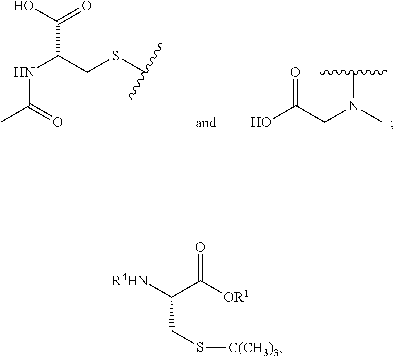
wherein
R¹ is selected from the group consisting of CH₃, CH₂CH₃, CH(CH₃)₂, CH₂-phenyl, and phenyl;
R⁴ is selected from the group consisting of H, C(O)R₂, and 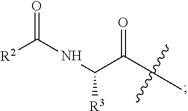
R² is selected from the group consisting of CH₃, CH₂CH₃, CH(CH₃)₂, CH₂-phenyl, and phenyl; and
R³ is selected from the group consisting of H, CH₃, CH₂-phenyl, CH(CH₃)₂, CH₂OH, 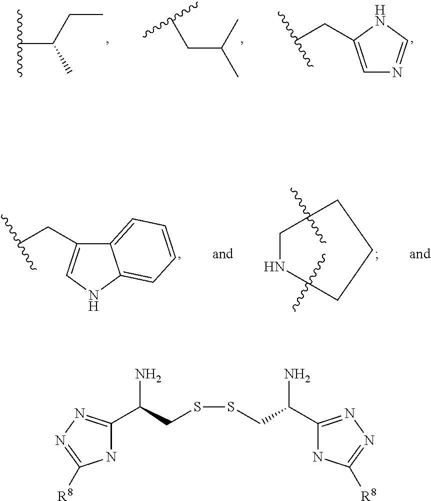
wherein R⁸ is selected from the group consisting of H, CH₃, CH₂CH₃, CH(CH₃)₂ and phenyl,
or pharmaceutically acceptable salts, esters, bioisosteres, enantiomers, diastereoisomers, mixtures of enantiomers/diastereoisomers, and prodrugs thereof.

4. A pharmaceutical composition for the treatment of Parkinson's Disease comprising the compound of claim 1 and a pharmaceutically acceptable carrier.

5. A pharmaceutical composition for the treatment of Parkinson's Disease comprising the compound of claim 2 and a pharmaceutically acceptable carrier.

6. A pharmaceutical composition for the treatment of Parkinson's Disease comprising the compound of claim 3 and a pharmaceutically acceptable carrier.
